(19) United States
(12) Patent Application Publication
(10) Pub. No.: US 2024/0023873 A1
(43) Pub. Date: Jan. 25, 2024

(54) PHRENIC NERVE INTEGRITY AND RAMPED-UP BURST

(71) Applicant: Covidien LP, Mansfield, MA (US)

(72) Inventors: Lilian KORNET, Eijsden (NL); Abhijit BHATTACHARYA, Hyderabad (IN)

(73) Assignee: Covidien LP, Mansfield, MA (US)

(21) Appl. No.: 18/347,667

(22) Filed: Jul. 6, 2023

Related U.S. Application Data
(60) Provisional application No. 63/391,430, filed on Jul. 22, 2022.

(51) Int. Cl. A61B 5/388; A61B 5/00

(52) U.S. Cl. CPC A61B 5/388; A61B 5/6847

(57) ABSTRACT

Aspects of this disclosure describe methods and systems for evaluating phrenic nerve integrity of a patient and a ramp-up pulse string for nerve stimulation. Integrity of a nerve may be assessed and therapy may be slowly introduced to the patient. To determine integrity, a nerve is electrically stimulated and a return signal is evaluated. For example, a time lapse and a band width of the return signal may provide information about nerve damage and a location of the nerve damage. In addition to assessing nerve integrity of a patient, a stimulation burst (e.g., an electrical stimulation) may be delivered as a ramped-up pulse string (e.g., a series of pulses with increasing voltages) to achieve smooth breathing. The voltages of a stimulation burst may be increased based on a target tidal volume for the patient. The stimulation burst may be delivered during an inhalation phase of a breath.

### CROSS-REFERENCE TO RELATED APPLICATIONS

This application claims the benefit of U.S. Provisional Application No. 63/391,430 filed 22 Jul. 2022, titled “Phrenic Nerve Integrity and Ramped-Up Burst,” which is incorporated herein by reference in its entirety.

### INTRODUCTION

Medical ventilator systems have long been used to provide ventilatory and supplemental oxygen support to patients. These ventilators typically include a connection for pressurized gas (air, oxygen) that is delivered to the patient through a conduit or tubing. As each patient may require a different ventilation strategy, modern ventilators can be customized for the particular needs of an individual patient. For example, several different ventilator modes or settings have been created to provide better ventilation for patients in different scenarios, such as mandatory ventilation modes, spontaneous ventilation modes, and assist-control ventilation modes. Ventilators monitor a variety of patient parameters and are well equipped to provide reports and other information regarding a patient's condition.

Long term external ventilation is typically provided to patients using positive pressure ventilation. Positive pressure ventilation is a form of artificial respiration in which a mechanical ventilator is used to deliver a controlled volume of gasses to the lungs of a patient. In contrast, in one form of negative-pressure ventilation, the diaphragm of a patient is caused to contract to cause the chest of the patient to expand during inspiration (thereby drawing air into the lungs), and the diaphragm is caused to relax to cause the chest to contract during exhalation (thereby forcing air out of the lungs). While lifesaving and valuable, positive pressure ventilation is non-physiological; that is, forcing air into the lungs is not the manner in which humans naturally breathe. Accordingly, the greater the positive pressure and/or the number of positive-pressure cycles, the more likely the patient will experience detrimental effects, such as an illness becoming more severe, ventilator-induced lung injury (VILI), acute respiratory distress syndrome (ARDS), ventilator-associated pneumonia (VAP), diaphragm dystrophy, and/or delay of ventilator weaning. These effects may increase an amount of time a patient is subjected to mechanical ventilation, leading to longer hospital stays and increased medical costs.

It is with respect to this general technical environment that aspects of the present technology disclosed herein have been contemplated. Furthermore, although a general environment is discussed, it should be understood that the examples described herein should not be limited to the general environment identified herein.

### SUMMARY

Among other things, aspects of the present disclosure include systems and methods for assessing phrenic nerve integrity and delivering a ramped-up stimulation burst. In an aspect, a method for determining phrenic nerve integrity is disclosed. The method includes positioning a set of electrodes inside a body of a patient at a location near a phrenic nerve and emitting a nerve stimulation pulse from the set of electrodes to stimulate the phrenic nerve. The method further includes receiving, at the set of electrodes, a return signal based on the nerve stimulation pulse, the return signal having a first signal and a last signal. Additionally, the method includes measuring a return signal band between the first signal and the last signal. Based on the return signal band, the method includes determining an integrity of the phrenic nerve.

In an example, positioning the set of electrodes inside the body includes: placing the set of electrodes at a first nerve depth of the phrenic nerve; measuring a first time lapse value between the nerve stimulation pulse and the first signal of the return signal at the first nerve depth, wherein the first time lapse value is less than a threshold value; placing the set of electrodes at a second nerve depth of the phrenic nerve; and measuring a second time lapse value between the nerve stimulation pulse and the first signal of the return signal at the second nerve depth, wherein the second time lapse value is at least the threshold value. In another example, based on the first time lapse and the second time lapse, the method further includes determining a presence of nerve damage of the phrenic nerve between the first nerve depth and the second nerve depth. In a further example, the set of electrodes is positioned inside of a vein or an esophagus of the patient. In yet another example, determining the integrity of the phrenic nerve includes determining, based on the return signal band, that nerve damage is located along at least one branch of the phrenic nerve.

In another aspect, a method for determining phrenic nerve integrity is disclosed. The method includes emitting a nerve stimulation pulse from a first electrode at a nerve depth to stimulate a phrenic nerve of a patient. Based on the nerve stimulation pulse, the method includes receiving a return signal at a second electrode at the nerve depth, wherein the return signal includes at least a first signal and a last signal, wherein a time lapse is defined between the nerve stimulation pulse and the first signal of the return signal, and wherein a return signal band is defined between the first signal and the last signal. Based on the time lapse and the return signal band, the method includes determining an integrity of the phrenic nerve. Additionally, the method includes determining treatment information for the patient, based on the integrity of the phrenic nerve.

In an example, the integrity of the phrenic nerve includes a location of nerve damage along the phrenic nerve. In another example, the phrenic nerve is a first phrenic nerve and the integrity is a first integrity, and wherein the method further includes: determining a second integrity of a second phrenic nerve; and determining a combined integrity of the first phrenic nerve and the second phrenic nerve based on the first integrity and the second integrity, wherein the treatment information is based on the combined integrity. In a further example, the treatment information includes one of: a feasibility of phrenic nerve stimulation therapy; the first integrity of the first phrenic nerve; the second integrity of the second phrenic nerve; or the combined integrity. In yet another example, the method further includes: displaying the treatment information.

In a further aspect, a method for ramping up nerve stimulation is disclosed. The method includes providing, from a stimulation device, a stimulation burst to a patient. The stimulation burst includes an initial stimulation pulse with an initial voltage. The stimulation burst also includes a final stimulation pulse with a final voltage. Additionally, the stimulation burst includes at least one middle stimulation pulse between the initial stimulation pulse and the final stimulation pulse, the middle stimulation pulse having at least one middle voltage greater than the initial voltage and less than the final voltage.

In an example, the initial stimulation pulse, the at least one middle stimulation pulse, and the final stimulation pulse are all emitted within less than three seconds. In another example, the stimulation burst is a first stimulation burst, the method further comprising: measuring a tidal volume delivered to the patient according to the first stimulation burst; comparing the measured tidal volume with a target tidal volume; and based on the comparison, delivering, from the stimulation device, a second stimulation burst including a second initial stimulation pulse with a second initial voltage and a second final stimulation pulse with a second final voltage, wherein the second final voltage is higher than the second initial voltage and higher than the final voltage of the first stimulation burst. In a further example, a slope defined by the initial voltage, the at least one middle voltage, and the final voltage is linear. In yet another example, the initial voltage is less than 30% of the final voltage.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 shows an anatomy of a human patient, including phrenic nerves.

FIG. 2 shows a stimulation device intravenously placed in the body of the patient of FIG. 1.

FIG. 3 shows a stimulation device placed in an esophagus in the body of the patient of FIG. 1.

FIG. 4 shows nerve depths of a right phrenic nerve and a left phrenic nerve in a body of a patient, including branch ends of the nerves on the diaphragm.

FIG. 5 shows a method for assessing integrity of a nerve.

FIG. 6 shows a method for placing electrodes near a nerve to determine a status of the nerve.

FIG. 7 shows another method for assessing integrity of a nerve.

FIGS. 8A-8D show graphical representations of return signals of a nerve over time as originating from a stimulation pulse.

FIG. 9 shows a graphical representation of a ramped-up stimulation burst voltage over time.

FIG. 10 shows a method for a ramped-up a stimulation burst to stimulate a nerve.

FIG. 11 is a diagram illustrating an example of a ventilator connected to a patient and a stimulation device.

FIG. 12 is a block-diagram illustrating an example of a ventilatory system.

FIG. 13 is an example stimulation system including a stimulation device, an application tool, and a connector.

FIG. 14 is another example stimulation system including a stimulation device, an application tool, and a connector.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently. In modern medical facilities, pressurized air and oxygen sources are often available from wall outlets, tanks, or other sources of pressurized gases. Accordingly, ventilators may provide pressure regulating valves (or regulators) connected to centralized sources of pressurized air and pressurized oxygen. The regulating valves function to regulate flow so that respiratory gases having a desired concentration are supplied to the patient at desired pressures and flow rates.

Approximately one third of patients in the intensive care unit depend on mechanical ventilation. With millions of people each year admitted to the intensive care unit, many people per year rely on mechanical ventilation. Additionally, respiratory illnesses such as SARS-CoV-2 (otherwise referred to as COVID-19) increase the number of individuals depending on mechanical ventilation. The amount of time a patient is assisted via mechanical ventilation may vary, as may be based on a lung condition of the patient. For example, approximately a third of patients with COPD or ARDS may seek assistance from a mechanical ventilator for longer than 4 days, with some patients requiring mechanical ventilation longer than seven days. Additionally, patients with sleep apnea may require mechanical ventilation while sleeping, over an extended period of time.

Weaning patients off of mechanical ventilation may be difficult. A patient's diaphragm muscles may begin to atrophy after as little as two days of ventilation. After a patient's diaphragm muscles begin to atrophy, a patient may require slow weaning to encourage the patient to breathe on their own (e.g., via the patient's diaphragm contracting unassisted by a mechanical ventilator). Increased time on a ventilator is associated with increased risk of infection, hyperventilation, hypoventilation, decreased venous return, and subsequent rehospitalization. Thus, minimizing a patient's time on a ventilator may be desirable for diaphragm health and/or to reduce the risk of infection and/or rehospitalization.

One solution is to exercise the patient's diaphragm muscles during ventilation to prevent or reduce the likelihood of diaphragm muscle atrophy and reduce the time that a patient is dependent on mechanical ventilation. To exercise a patient's diaphragm, the phrenic nerve can be stimulated with an electrical current. The electrical current to stimulate the phrenic nerve may be provided by electrodes producing a nearby electrical current. The electrodes may be positioned inside of the body of the patient close enough to the phrenic nerve to stimulate the phrenic nerve. For example, electrodes may be positioned on a device that is insertable into the body of the patient. In one example, electrodes may be positioned on a lead insertable into a blood vessel (e.g., vein, artery, arteriole, capillary, venule, etc.) near a phrenic nerve or in a structure of a catheter insertable into an esophagus near a phrenic nerve.

Although pacing therapy of a phrenic nerve (sometimes referred to as phrenic nerve stimulation therapy, pacing therapy, or diaphragm pacing) has many benefits (e.g., preventing or reversing diaphragm muscle-disuse atrophy, maintaining diaphragmatic endurance, facilitating weaning of patients from mechanical ventilation, etc.), some patients may not have enough nerve integrity to benefit from this form of therapy. For example, a phrenic nerve may be broken or damaged between the electrodes and the diaphragm. If nerve damage is present between the stimulation and the diaphragm, then stimulation of the phrenic nerve may not activate the diaphragm a sufficient amount or at all. Phrenic nerve integrity is often tested at the clavicle region, which may produce false negatives (e.g., false indicators that phrenic nerve therapy is unavailable) due to distance between the clavicle and the nerves and may result in dismissing many patients who could otherwise benefit from phrenic nerve stimulation therapy. Additionally, when beginning to administer nerve stimulation therapy, the system may be unprepared for certain amounts of stimulation, which could sometimes cause hiccups or other discomfort to the patient.

Aspects of this disclosure describe evaluating phrenic nerve integrity of a patient and a ramp-up pulse string for nerve stimulation. In particular, prior to administering phrenic nerve stimulation therapy, integrity of a nerve may be assessed and therapy may be slowly introduced to the patient. The integrity of a nerve (e.g., a phrenic nerve) may be evaluated using electrically evoked compound action potentials (eCAP). The nerve is electrically stimulated and a return signal may be measured to determine integrity. For example, a time lapse of the return signal (e.g., a time lapse value) and a return signal band of the return signal may provide information about nerve damage and where the damage is located along the nerve. Depending on the location of damage along the nerve, stimulation may be provided between the location of damage and the diaphragm. In addition to assessing nerve integrity of a patient, a stimulation burst (e.g., an electrical stimulation) may be delivered as a ramped-up pulse string (e.g., a series of pulses with increasing voltages) to achieve smooth breathing. The voltages of the stimulation burst may be increased based on a target tidal volume for the patient. By ramping up stimulation of a nerve, a patient may experience less discomfort from a sudden electrical signal introduced into their body (e.g., reduce the likelihood of hiccups, reduce strain on the diaphragm, ease into any physiological changes cause by stimulation of a nerve, etc.).

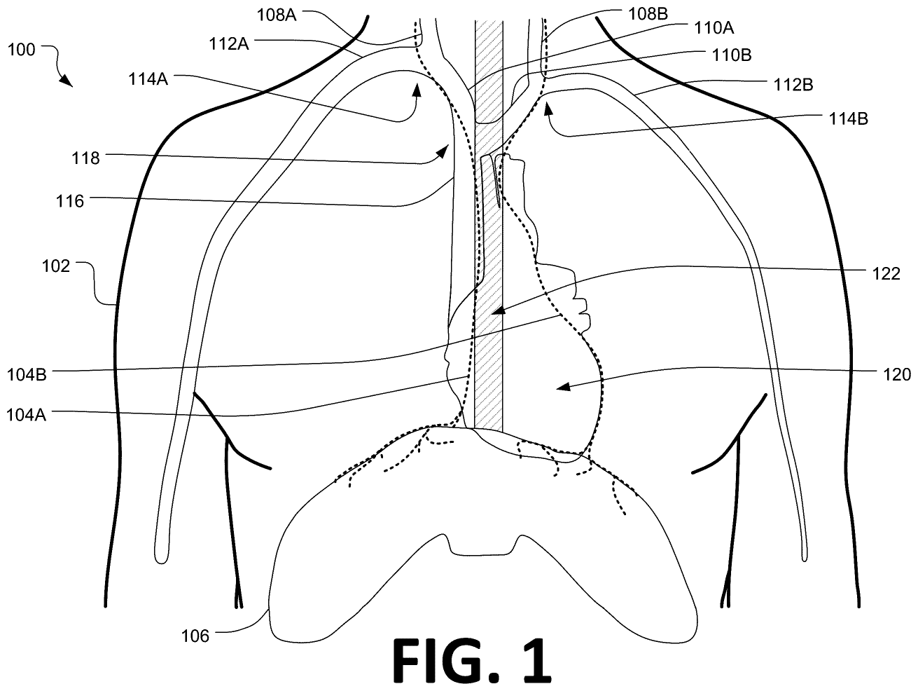

FIG. 1 shows an anatomy of a human patient 100, including phrenic nerves (i.e., right phrenic nerve 104A and left phrenic nerve 104B). The body 102 of the patient 100 includes a right phrenic nerve 104A, a left phrenic nerve 104B, a diaphragm 106, a right internal jugular vein 108A, a left internal jugular vein 108B, a right brachiocephalic vein 110A, a left brachiocephalic vein 110B, a right subclavian vein 112A, a left subclavian vein 112B, a right jugular-brachiocephalic junction 114A, a left jugular-brachiocephalic junction 114B, a superior vena cava (SVC) 116, an SVC junction 118, a heart 120, and an esophagus 122. It should be appreciated that, although not shown, the body 102 of the patient 100 contains other anatomical structures, including a stomach fed by the esophagus 122.

The right phrenic nerve 104A and the left phrenic nerve 104B originate from the spinal cord in the neck region (C3-C5 cervical vertebral region). The right phrenic nerve 104A extends through the body 102 from the right side of the neck region between the right lung and the heart 120 to the right side of the diaphragm 106. The left phrenic nerve 104B extends through the body 102 from the left side of the neck region between the left lung and the heart 120 to the left side of the diaphragm 106. The right phrenic nerve 104A and the left phrenic nerve 104B may each, independently, cause muscle movement of e the diaphragm 106 using an electrical signal running down the nerve. The phrenic nerve is “stimulated” when the phrenic nerve sends an electrical signal to the diaphragm 106. To “effectively stimulate” the phrenic nerve, one or more muscles of the diaphragm 106 move (such as stiffening or contracting) due to the electrical signal sent from the phrenic nerve. The electrical signal to stimulate the phrenic nerve may originate naturally from the brain or may be provided artificially (e.g., by a nearby electrical current, and resulting magnetic field, from electrodes on a stimulation device). For example, the right phrenic nerve 104A and/or the left phrenic nerve 104B may be artificially stimulated by a magnetic field resulting from an electrical current between one or more electrodes on a stimulation device (such as an intravenous stimulation device or an esophageal stimulation device). Examples of stimulating one or both phrenic nerves via an intravenous stimulation lead are provided in U.S. patent application Ser. No. 17/039,115, titled “Intravenous Phrenic Nerve Stimulation Lead,” and filed on Sep. 30, 2020, which is incorporated herein by reference in its entirety. Additionally, examples of stimulating one or both phrenic nerves via an esophageal balloon catheter are provided in U.S. Patent Application No. 63/241,747, titled “Phrenic Nerve Stimulation with Mechanical Ventilation,” and filed on Sep. 8, 2021, which is incorporated herein by reference in its entirety. As an artificial magnetic field is created near the phrenic nerve, transmission fibers of the phrenic nerve are excited to create movement of muscles in the diaphragm. For the magnetic field to effectively stimulate the phrenic nerve, the one or more electrodes carrying the electrical current should be placed near, adjacent, or proximate the right phrenic nerve 104A and/or the left phrenic nerve 104B.

The electrical signal sent to the diaphragm 106 from the right phrenic nerve 104A and/or the left phrenic nerve 104B causes movement of one or more muscles of the diaphragm 106. Muscle movement of the diaphragm may cause expansion of the lungs of the patient. For example, stimulation of the right phrenic nerve 104A causes one or more muscles on a right portion of the diaphragm 106 to move, thereby causing expansion of one or both lungs of the patient 100. In another example, stimulation of the left phrenic nerve 104B causes one or more muscles on a left portion of the diaphragm 106 to move, thereby causing expansion of one or both lungs of the patient 100. Stimulation of only one of the phrenic nerves (i.e., right phrenic nerve 104A or left phrenic nerve 104B) may cause muscle movement on both sides of the diaphragm, due to the collective nature of the diaphragm muscles (e.g., stimulating muscles on one side will also move some of the muscles on the other side). For some patients, stimulation of only one of the phrenic nerves is sufficient, while other patients may require or desire stimulation of both phrenic nerves. Examples of stimulating one or both phrenic nerves to obtain tidal volumes are provided in U.S. patent application Ser. No. 16/888,960, titled “Achieving Smooth Breathing by Modified Bilateral Phrenic Nerve Pacing,” and filed on Jun. 1, 2020, which is incorporated herein by reference in its entirety.

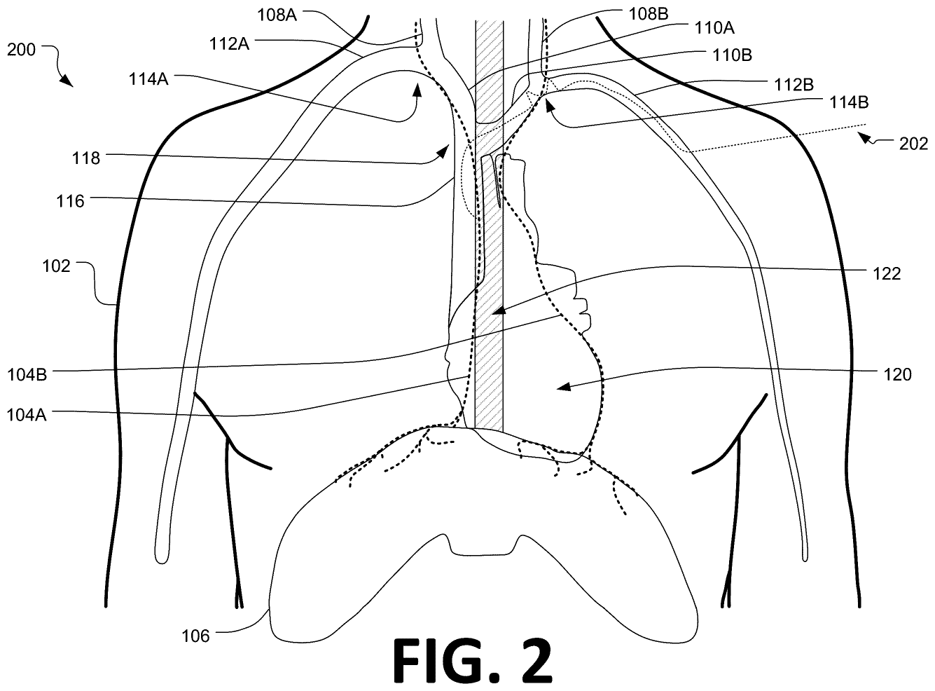

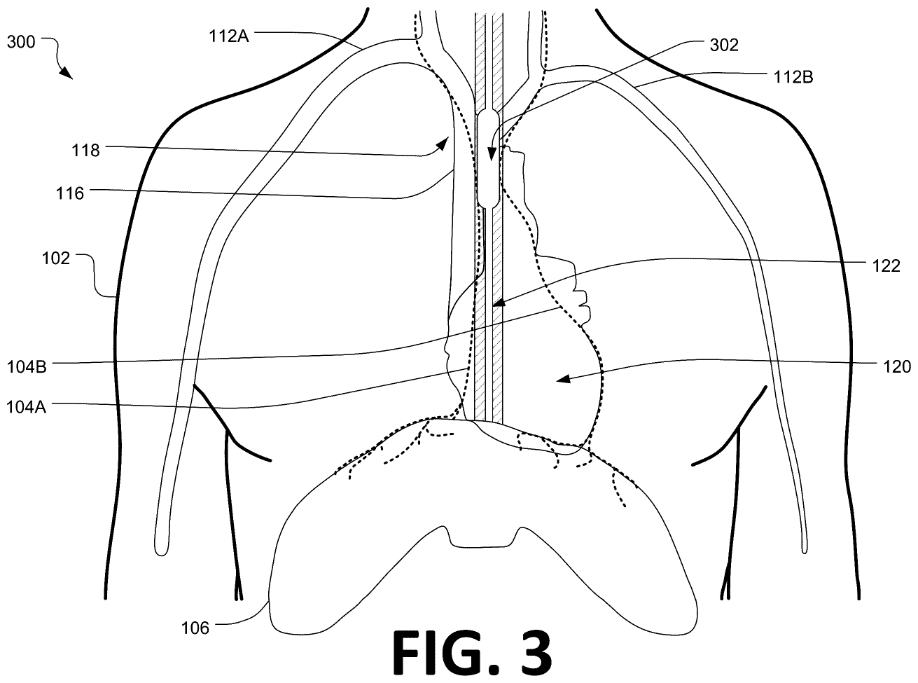

In an example, a phrenic nerve (e.g., the right phrenic nerve 104A and/or left phrenic nerve 104B) may be stimulated by a magnetic field caused by a current between two or more electrodes. The electrodes may be positioned inside of the body of a patient at a location where a phrenic nerve is inside the resulting magnetic field. In an example, the electrodes may be coupled to a stimulation device to facilitate introduction into the body. For instance, electrodes may be placed intravenously (e.g., via an intravenous stimulation lead) or esophagealy (e.g., via a stimulation device in an esophagus 122 of the patient 100). An intravenous stimulation lead is described with respect to FIG. 2. An esophageal stimulation device with electrodes is described with respect to FIG. 3.

FIG. 2 shows a stimulation device 202 in the form of a stimulation lead 402 intravenously placed in the body 102. The stimulation device 202 includes at least one set of electrodes capable of stimulating a nerve (e.g., right phrenic nerve 104A or left phrenic nerve 104B) from inside of a vein of the patient 200 (e.g., from inside at least one of a right internal jugular vein 108A, a left internal jugular vein 108B, a right brachiocephalic vein 110A, a left brachiocephalic vein 110B, a right subclavian vein 112A, a left subclavian vein 112B, a right jugular-brachiocephalic junction 114A, a left jugular-brachiocephalic junction 114B, a superior vena cava (SVC) 116, an SVC junction 118). The stimulation device 202 may include features or components to secure the stimulation device 202 inside of a vein. For example, the stimulation device 202 may include one or more deformable segments and one or more elongate segments. Electrodes may be placed on deformable segments and/or elongated segments of the stimulation device 202.

The stimulation lead may be implanted in the body 102. In the example shown in FIG. 2, at least a portion of the stimulation device 202 is positioned in the left subclavian vein 112B near the left jugular-brachiocephalic junction 114B, proximate the left phrenic nerve 104B, as secured by a deformable segment inside the left subclavian vein 112B. Although the stimulation device 202 is shown as secured in the left subclavian vein 112B, it should be appreciated that the stimulation device 202 may be positioned in, and secured in, any blood vessel in the body 102 that runs proximate a phrenic nerve (e.g., the right internal jugular vein 108A, the left internal jugular vein 108B, the right brachiocephalic vein 110A, the left brachiocephalic vein 110B, the right subclavian vein 112A, the left subclavian vein 112B, the right jugular-brachiocephalic junction 114A, the left jugular-brachiocephalic junction 114B, SVC 116, SVC junction 118, carotid arteries, subclavian arteries, aorta, etc.). The stimulation device 202 may be employed to stimulate one or both phrenic nerves for pacing of muscles of the diaphragm 106.

The stimulation device 202 is capable of providing voltage to one or more electrodes coupled to the stimulation device 202 from a power source. Attributes (e.g., voltage, frequency, pulse length, etc.) of each electrode of the stimulation device 202 may be individually addressable and controllable by a controller (such as a PCB). In an example, a clinician may control the nerve pacing of the stimulation device 202 via a controller, and observe the resultant ventilatory efforts of the patient at the ventilator. The controller may be a component of the ventilator, or may be a separate device. In an example where the controller is integrated into the ventilator, the power source is provided by the ventilator. In an example where the controller is a separate device, the controller may be associated with a display and user interface to allow viewing or selecting of electrode attributes.

FIG. 3 shows a stimulation device 302 placed in an esophagus 122 in a body 102 of a patient 300. The stimulation device 302 shown in FIG. 3 is a balloon catheter including a catheter, an inflatable balloon, and electrodes (not shown) capable of stimulating one or more phrenic nerves 104A, 104B.

The stimulation device 302 may be placed and secured in the body 102 by inflating the inflatable balloon. The stimulation device 302 may be placed along different depths of the esophagus 122, such as proximate a phrenic nerve (e.g., right phrenic nerve 104A or left phrenic nerve 104B, or both) and/or proximate the diaphragm 106. In the example shown in FIG. 3, at least a portion of the inflatable balloon (which is in an inflated configuration to secure the stimulation device 302 in the esophagus 122) of the stimulation device 302 is placed in the upper thoracic esophagus or in the mid-thoracic esophagus proximate both the right phrenic nerve 104A and the left phrenic nerve 104B. In an alternative position, at least a portion of the inflatable balloon of the stimulation device 302 is placed in the lower thoracic esophagus or abdominal esophagus proximate both the right phrenic nerve 104A and the left phrenic nerve 104B and/or extensions of the right phrenic nerve 104A and/or the left phrenic nerve 104B along the diaphragm 106.

Similar to the example stimulation device 202 shown in FIG. 2, the stimulation device 302 is capable of providing voltage to one or more electrodes coupled to the stimulation device 302 from a power source. Attributes of each electrode along the stimulation device 302 may be individually addressable and controllable by a controller (such as a PCB). The controller may be a component of a ventilator, or may be a separate device. In an example where the controller is integrated into the ventilator, the power source is provided by the ventilator. In an example where the controller is a separate device, the controller may be associated with a display and user interface to allow viewing or selecting of electrode attributes.

For both of the example stimulation devices 202, 302 shown in FIGS. 2-3, a magnetic field produced by the electrodes may be extended. For example, an external pad may be placed on or near the skin of the patient, external to the patient, while a stimulation device 202, 302 is actively placed inside of the body 102. The external pad may include one or more electrodes. The external pad may be moved relative to the body 102 of the patient 100 and/or relative to the stimulation device 202, 302 to provide more or less stimulation of a phrenic nerve of a patient.

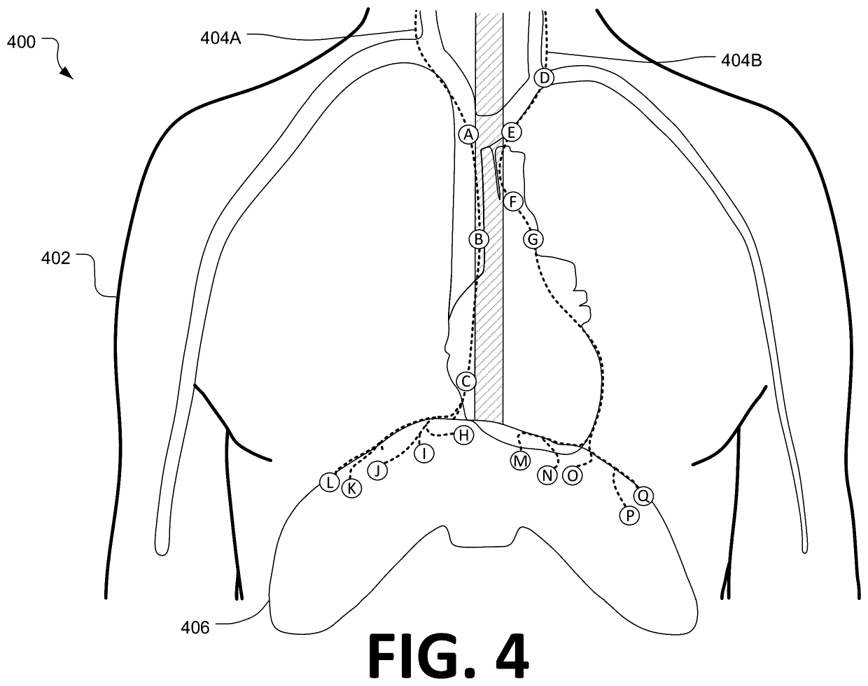

Depending on the stimulation device 202, 302, the right phrenic nerve 104A and/or the left phrenic nerve 104B may have a maximum depth (e.g., as may be measured from the neck or clavicle of the patient downwards towards the diaphragm) capable of being stimulated by the stimulation device 202, 302. In an example where the stimulation device 202 is placed intravenously, the maximum depth for stimulation of both the right phrenic nerve 104A and the left phrenic nerve 104B may be just above the junction of the SVC and the heart (e.g., as shown by nerve depth B for the right phrenic nerve and nerve depth G for the left phrenic nerve in FIG. 4).

Alternatively, in an example where the stimulation device 302 is placed in an esophagus 122 of the patient 100, the maximum depth for stimulation of the right phrenic nerve 104A may be deeper than the left phrenic nerve 104B. This may be in part due to placement of a patient's right phrenic nerve 104A and left phrenic nerve 104B with respect to the esophagus 122. For instance, as shown at least in FIG. 3, the right phrenic nerve 104A may travel proximate the esophagus 122 all the way to the diaphragm 106 and thus may be stimulated from the esophagus 122 by a stimulation device 302 at any nerve depth (e.g., no maximum depth for stimulating the right phrenic nerve 104A and may be stimulated at nerve depth C shown in FIG. 4, or below). Alternatively, the left phrenic nerve 104B may not travel proximate the esophagus 122 at all depths. As shown in FIG. 3, the left phrenic nerve 104B may diverge in a direction away the esophagus 122 above the junction of the SVC with the heart. Thus, a maximum depth for stimulation of the left phrenic nerve 104B with an esophageal stimulation device 302 may be proximate the SVC and above the heart (e.g., as shown by nerve depth F in FIG. 4).

FIG. 4 shows nerve depths A-Q of a right phrenic nerve 404A and a left phrenic nerve 404B in a body 102 of a patient 400, including branch ends H-Q of the nerves on the diaphragm 406. As described herein, each of the right phrenic nerve 404A and the left phrenic nerve 404B may have maximum depths at which they may be stimulated, depending on the type of stimulation device. For example, a stimulation device placed intravenously may be capable of stimulating the right phrenic nerve 404A between nerve depth A (e.g., near the top of the SVC near the junction of the right and left brachiocephalic veins) and nerve depth B (e.g., just above the heart). The intravenously-placed stimulation device may be capable of stimulating the left phrenic nerve 404B between nerve depth D (e.g., in the left brachiocephalic vein) and nerve depth G (e.g., just above the heart).

In another example, a stimulation device placed in an esophagus may be capable of stimulating the right phrenic nerve 404A between nerve depth A (e.g., near the top of the SVC near the junction of the right and left brachiocephalic veins) and nerve depth C (e.g., just above the diaphragm 406). The esophageal stimulation device may be capable of stimulating the left phrenic nerve 404B between nerve depth E (e.g., near the top of the SVC near the junction of the right and left brachiocephalic veins) and nerve depth F (e.g., along the SVC above the heart).

As shown and described, the right phrenic nerve 404A may have one or more branches extending along the diaphragm 406. Each of the right branches includes a branch end H-L at which the right phrenic nerve 404A terminates along the diaphragm 406. Although at least five branch ends H-L are described with respect to the right phrenic nerve 404A, any number of branches and branch ends are appreciated for the right phrenic nerve 404A, such as three branches or branch ends, four branches or branch ends, five branches or branch ends, or six or more branches or branch ends. Similar to the right phrenic nerve 404A, the left phrenic nerve 404B may have one or more branches extending along the diaphragm 406. Each of the left branches includes a branch end M-Q at which the left phrenic nerve 404B terminates along the diaphragm 406. Any number of branches and branch ends are appreciated for the left phrenic nerve 404B.

Nerve damage may occur at any nerve depth of the right phrenic nerve 404A or the left phrenic nerve 404B. For example, nerve damage or a break in a nerve may occur proximate to (close to) the diaphragm 406, such as along any branch of a nerve. For instance, damage may occur between nerve depth C and branch ends H-L along the right phrenic nerve 404A shown in FIG. 4). Additionally or alternatively, nerve damage may occur upstream of (away from) the diaphragm 406, such as above the branches of a nerve. Depending on a location of nerve damage along a phrenic nerve and a maximum stimulation depth of a stimulation device (as described above), stimulation of the phrenic nerve may or may not be possible with the stimulation device. If one or more electrodes may be placed at a nerve depth below the damage or break in the phrenic nerve, then stimulation of the diaphragm via the nerve may be an available treatment option. If, alternatively, nerve damage or a break in the nerve is below the one or more electrodes, then stimulation of the diaphragm via the nerve may not be an available treatment option. For example, if nerve damage is located above a maximum stimulation depth for a stimulation device (e.g., a maximum stimulation depth for an intravenous stimulation device at nerve depth B for the right phrenic nerve and nerve depth G for the left phrenic nerve, a maximum stimulation depth for an esophageal stimulation device at nerve depth C for the right phrenic nerve and nerve depth F for the left phrenic nerve), then phrenic nerve stimulation (PNS) of the diaphragm is possible (e.g., feasible). Alternatively, if nerve damage is located at or below the maximum stimulation depth for a stimulation device, then PNS of the diaphragm may not be possible (e.g., not feasible). Examples of nerve damage location and stimulation feasibility is summarized in Table 1, below.

TABLE 1

| Phrenic | Nerve | Stimulation | |
|---|---|---|---|
| Nerve | Damage | Device | PNS |
| Side | Location | Type | Possible? |
| Right | Above nerve depth A | Intravenous | Yes |
| | | Esophageal | Yes |
| | At or below nerve depth A | Intravenous | Yes |
| | and above nerve depth B | Esophageal | Yes |
| | At or below nerve depth B | Intravenous | No |
| | and above nerve depth C | Esophageal | Yes |
| | Below nerve depth C | Intravenous | No |
| | | Esophageal | No |
| Left | Above nerve depth D | Intravenous | Yes |
| | At or below nerve depth D | Esophageal | Yes |
| | and above nerve depth E | Intravenous | Yes |
| | At or below nerve depth E | Esophageal | Yes |
| | and above nerve depth F | Intravenous | Yes |
| | At or below nerve depth F | Esophageal | Yes |
| | and above nerve depth G | Intravenous | Yes |
| | Below nerve depth G | Esophageal | No |
| | | Intravenous | No |
| | | Esophageal | No |

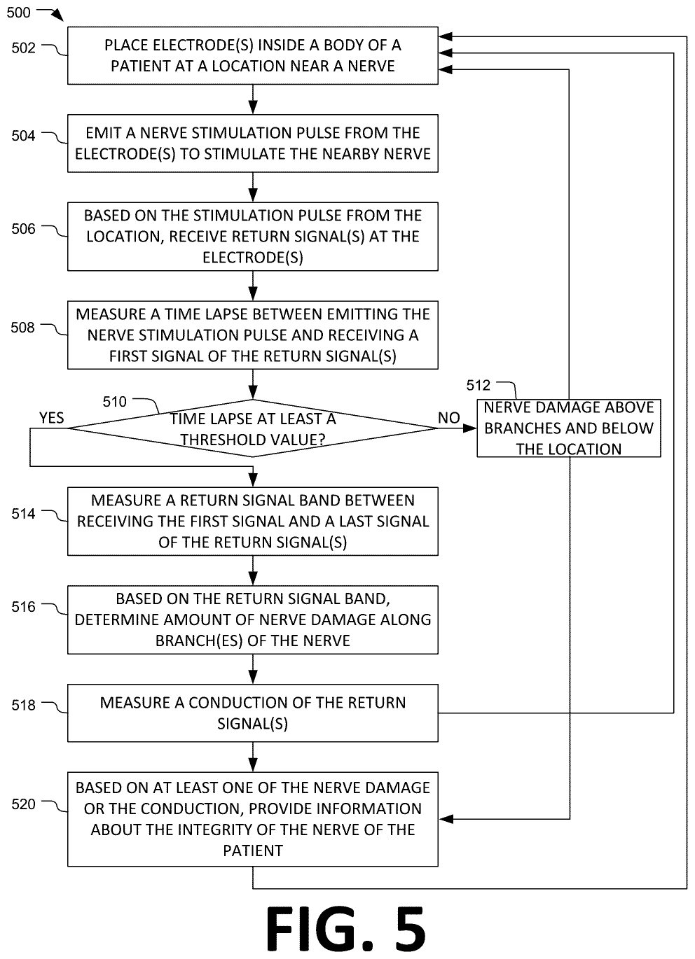

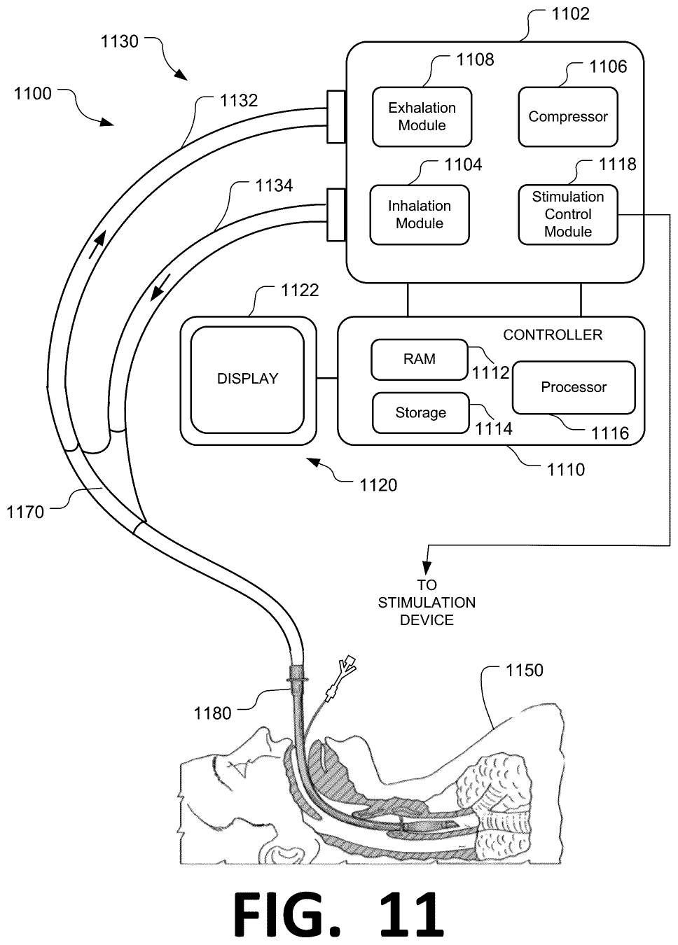

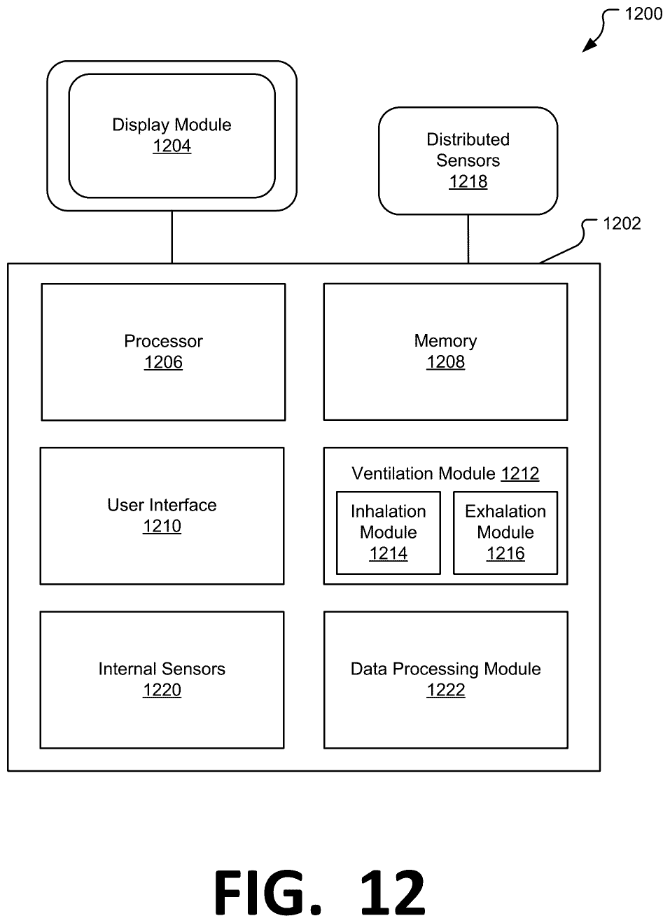

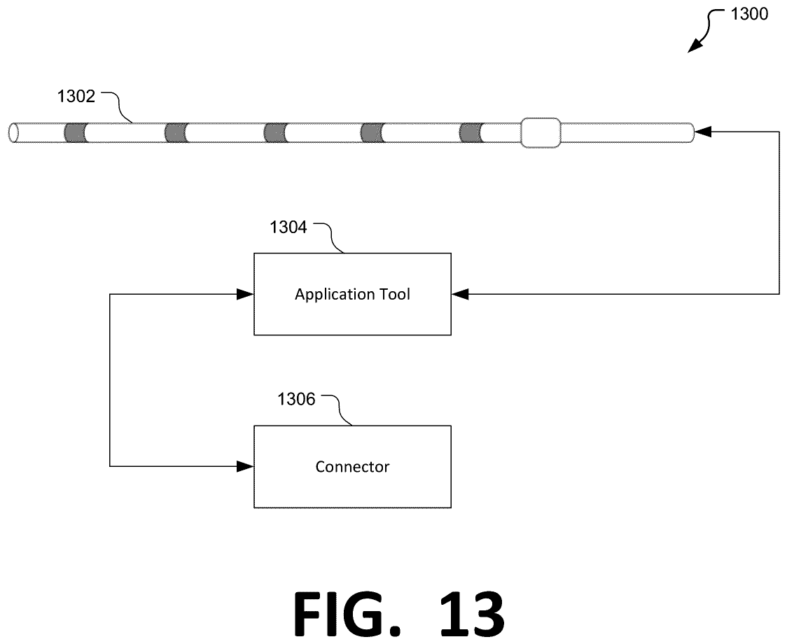

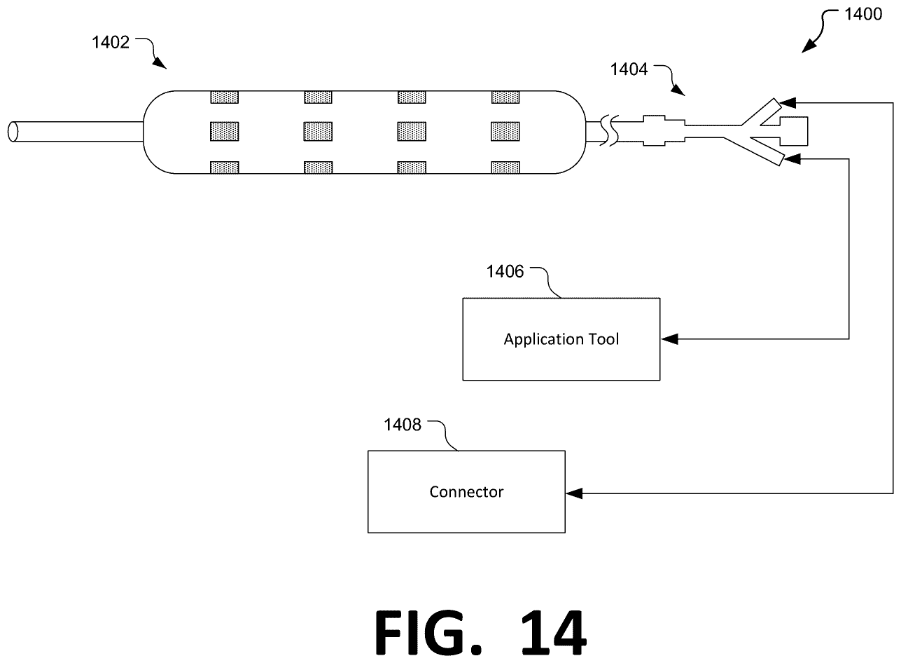

FIGS. 5-7 and 9 show example methods according to the disclosed technology. The example methods include operations that may be implemented or performed by the systems and devices disclosed herein. For example, the stimulation device 202 depicted in FIG. 2, the stimulation device 302 depicted in FIG. 3, the ventilator 1100 depicted in FIG. 11, the ventilatory system 1200 depicted in FIG. 12, the system 1300 depicted in FIG. 13, and/or the system 1400 depicted in FIG. 14 may perform the operations described in the methods. In addition, instructions for performing the operations of the methods disclosed herein may be stored in a memory of the various systems (e.g., system memory 1112 described in FIG. 11 and/or memory 1208 described in FIG. 12).

FIG. 5 shows a method 500 for assessing integrity of a nerve (e.g., phrenic nerves 104A, 104B, 404A, 404B). If a nerve is damaged or has limited integrity, then treatment options or treatment information for a patient may be modified or informed, based on the damage and/or integrity.

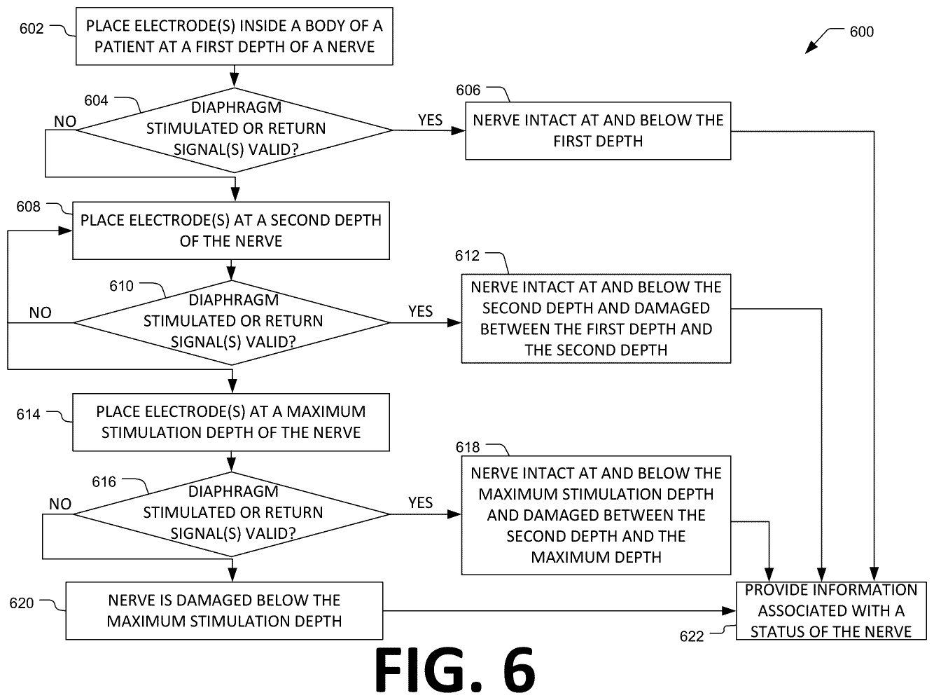

At operation 502, one or more electrodes are placed inside a body of a patient at a location near a nerve. As described herein, a stimulation device placed in a body of a patient may include one or more electrodes capable of stimulating a nearby nerve. For instance, a stimulation device may be placed intravenously (e.g., stimulation device 202 in FIG. 2) or in an esophagus (e.g., stimulation device 302 in FIG. 3). Placement of the electrodes (e.g., which may also include placement of a stimulation device coupled with the electrodes), may be based on if and where a nerve is intact between the placement location and the diaphragm. For example, electrodes may be positioned at least at a nerve depth where the nerve is intact between the position and the diaphragm, where possible. As described above, depending on the stimulation device and the particular nerve, the maximum nerve depth may vary. Placement of the electrodes is further described at least with respect to FIG. 6.

At operation 504, a nerve stimulation pulse is emitted from at least one electrode to stimulate the nearby nerve. The stimulation pulse may have attributes, such as an amplitude, voltage, frequency, duration, etc. In an example, one electrode emits the stimulation pulse. An electrode emitting a stimulation pulse may, for a short period of time, be blind to sensing or receiving any return signals associated with the stimulation pulse. Thus, a different electrode at the same nerve depth as the emitting electrode may be used at operation 506 when receiving one or more return signals. The emitted stimulation pulse may stimulate the nerve via an electrically evoked compound action potential (eCAP), which is associated with the synchronous firing of nerve fibers along a nerve.

At operation 506, one or more return signals are received at the receiving electrodes, based on the stimulation pulse from the emitting electrodes. As described above, the emitting electrode and the receiving electrode may be the same electrode or different electrodes. The return signals are caused by a reflection or bounce-back at ends of, or breaks in, the nerve stimulated by the stimulation pulse.

At operation 508, a time lapse or latency is measured between emitting the nerve stimulation pulse and receiving a first return signal of the return signals. The time lapse or latency may otherwise be described as the round-trip time of the stimulation pulse being emitted, reflecting back, and first being received. In an example, the stimulated nerve may be broken prior to branching, which may result in one return signal being received with a short time lapse (e.g., a short latency). In another example, the stimulated nerve may be broken along the diaphragm at one of the branches. This may cause multiple return signals (e.g., one for each branch upstream of the break and one for the break itself). The first signal returned may have a longer time lapse, relative to the prior example of a break above the diaphragm, and a longer duration or band due to multiple return signals (e.g., as compared to one return signal in the prior example). In a further example, the stimulated nerve and its branches may be intact and multiple return signals may be received (e.g., one for each branch), where the first signal returned has a longer time lapse, relative to nerve damage upstream of the diaphragm, which may be the same time lapse as the prior example where a break was located along a branch. In this instance, the return signals span a longer duration because each branch is intact and reflects a stimulation pulse at each branch end.

Stated another way, nerves with multiple intact branches reflect multiple return signals (e.g., one for each branch, as reflected at each branch end). Nerves with a break along the branches may also reflect multiple return signals (e.g., one for each branch upstream of the break, as reflected at each branch end, and one for the break itself). If the break is located along the first branch, from which all other branches originate, then one signal may be reflected (e.g., for the break itself). If the break is above the diaphragm, then one return signal may be reflected (e.g., at the break itself). Breaks or nerve damage above the diaphragm has a short latency (e.g., less than a threshold) and breaks at or below the diaphragm have a long latency (e.g., greater than or equal to a threshold).

Multiple return signals may be received at different time points due to the variations in length of the branches. Where one branch is short or damaged, but another branch is long and intact, the time interval (e.g., return signal band or duration) between the first reflected signal (e.g., from the short or damaged branch) and the last reflected signal (e.g., from the longest branch) will be greater than if all the branch reflection points (e.g., at a branch end or at a break) are positioned at similar lengths.

At determination 510, a determination is made as to whether the time lapse for the first return signal is at least a threshold value. A threshold value may be based on a nerve depth of the electrodes emitting the stimulation pulse. For example, if the stimulating electrodes are placed to stimulate at a deep nerve depth (e.g., nerve depth C in FIG. 4), the threshold value may be smaller due to proximity of the electrodes with the diaphragm. Alternatively, if the stimulating electrodes are placed to stimulate at a shallow nerve depth (e.g., nerve depth A or nerve depth D in FIG. 4), the threshold value may be larger due to distance between the electrodes and the diaphragm. Stated alternatively, a shallower depth of stimulation has a longer path for the signal to travel and therefor requires additional travel time (e.g., as associated with a longer threshold value) for return signals to reflect due to distance between the electrodes and the branch ends. A deeper depth of stimulation is associated with shorter time intervals (e.g., as associated with a shorter threshold value) for return signals to reflect due to the closer proximity of the electrodes and the branch ends.

A velocity of the return signals may be estimated, based on the time lapse (e.g., the time difference between a signal being sent and received) and the nerve depth. For example, the velocity of a signal may be determined based on an estimated distance the signal travels (e.g., double the distance between the nerve depth and a reflection point, such as branches along the diaphragm) divided by the time lapse of the signal. The velocity of the signal may be associated with a type of fibers of the nerve. For example, different velocity ranges may indicate a type of fiber of the nerve, such as A-fibers and C-fibers, and if one type of fiber is more or less intact than another.

If the time lapse is less than the threshold value, the method 500 flows “NO” to operation 512. At operation 512, nerve damage is determined to be present. Additionally, the nerve damage is determined to be located above the branches of the nerve and below the location of the electrodes (e.g., the damage is between the emitting electrode and the diaphragm) For instance, a return signal that is received too quickly may be due to a break or damage in the nerve that causes the return signal to reflect from the broken end of the nerve at the damaged location.

If, alternatively, the time lapse is at least the threshold value, the method 500 flows “YES” to operation 514. At operation 514, a return signal band is measured between receiving the first signal of the return signals and receiving a last signal of the return signals. For instance, the time interval between the time first return signal is received to the time the last return signal is received is the return signal band.

As described herein, multiple return signals may be received. For example, if a nerve has more than one branch end (e.g., from one or more branches, such as branch ends H-Q in FIG. 4), a return signal may be received for each branch end. A branch end may reflect a return signal if upstream of the branch end (e.g., between the electrodes and the branch end) is intact. Alternatively, a return signal may be reflected from any break along the nerve or branch. For instance, if a first branch with two branch ends is intact and a second branch is broken prior to any of its three branch ends, then three return signals may be received (one for each of the two branch ends along the intact branch and one for the break along the broken branch).

A quantity of return signals may also be determined or calculated. The quantity of return signals may be associated with a width of the return signal band, due to differences in timing of return signals from branch ends. For example, several intact branches may cause multiple return signals spanning a range of return times and thus creating a wide and/or populous return signal band (e.g., multiple return times ranging from 0.2 seconds to 0.7 seconds). Alternatively, one or more broken branches, or breaks relatively upstream along a branch, may cause fewer return signals spanning a shorter range (e.g., in some examples the range may be one signal or more than one signal) of return times and creating a narrow and/or sparse return signal band (e.g., one signal at 0.5 seconds). Additionally, an expected quantity and expected amplitude of the multiple return signals (e.g., the expected return signals could be determined based on a control) may be compared with the measured return signals. If a return signal is missing or has a smaller amplitude than expected, the difference could indicate that one type of fiber is damaged.

At operation 516, an amount of nerve damage along one or more branches of the nerve is determined based on the return signal band. As indicated herein, a width (e.g., wide or narrow) and/or population (e.g., populous or sparse) of the return signal band may therefore be an indicator of branch damage of the stimulated nerve, with a narrower return signal band being associated with more nerve damage.

At operation 518, a conduction of the return signals is measured. A conduction of the return signals may be measured based on an amplitude of the eCAP received. Although the time lapse of the return signals and the width of the return signal band may indicate a quantity of branches of a nerve being intact, the conduction may be used to determine the integrity of the intact branches. Higher conduction indicates better integrity. Better integrity of a nerve may be associated with larger tidal volumes of the patient in response to stimulation of the nerve. Alternatively, lower conduction indicates worse integrity and less tidal volume caused by stimulation of the nerve. If integrity of one or more nerves (e.g., the right phrenic nerve and/or the left phrenic nerve) is too low, then phrenic nerve stimulation therapy may not be desirable or feasible for the patient.

At operation 520, information about the integrity of the nerve of the patient is provided, based on at least one of the nerve damage (e.g., as determined at operation 512, 516) or the conduction (e.g., as determined at operation 518). The information may be provided to a clinician or doctor to determine a treatment plan for the patient, which may include whether phrenic nerve stimulation therapy is a viable or desirable treatment option for the patient. In an example, the information may be provided (e.g., displayed) on a user interface of a device, such as a ventilator, a stimulation device, external display, etc.

Operations 502-512 or operations 502-518 or operations 502-520 may repeat as required or desired. For example, the repeating operations may repeat for a different nerve. In an example, two nerves may be stimulated simultaneously. Alternatively, the operations may repeat for a different location of the electrodes for a same or different nerve to determine nerve function and integrity at different points along the nerve.

FIG. 6 shows a method 600 for placing electrodes near a nerve to determine a status of the nerve (e.g., intact or broken). At operation 602, electrodes are placed inside a body of a patient at a first depth of a nerve. The first depth may be the shallowest depth possible based on a stimulation device carrying the electrodes. For example, for a right phrenic nerve (e.g., phrenic nerve 104A) the first depth may be near a top of an SVC of a patient (e.g., nerve depth A) for a stimulation device placed in a vein or an esophagus of the patient. For a left phrenic nerve (e.g., phrenic nerve 104B), the first depth may be either in the left brachiocephalic vein for an intravenous stimulation device (e.g., nerve depth D) or near a top of an SVC (e.g., nerve depth E) for a stimulation device placed in the esophagus.

At determination 604, it is determined if, at the first depth of the nerve, the diaphragm is stimulated and/or return signals are consistent with stimulation of the diaphragm (e.g., the return signals indicate the nerve is intact). Movement of the chest of the patient may be caused by diaphragm stimulation and may be determined based on sight or feel. As described previously, intactness of a nerve and its branches may be determined based on signal attributes of the return signals, such as time lapse and return signal band. If a time lapse and a return signal band are satisfy threshold values (e.g., are greater than or equal to the threshold values), then the return signals may be determined to be valid (e.g., the nerve is intact and/or the return signals are consistent with stimulation of the diaphragm). In an example where a diaphragm is stimulated at the diaphragm, rather than upstream of the diaphragm along a phrenic nerve, a fiber type may not be identifiable because the pathway of the return signals may not be a constant distance from the electrodes due to movement of the diaphragm.

If it is determined that, at the first depth of the nerve, the diaphragm is stimulated and/or the return signals are valid (e.g., the nerve is intact from the stimulation location to the diaphragm), then the method 600 flows “YES” to operation 606. At operation 606, it is determined that the nerve is intact at and below the first depth of the nerve.

If, alternatively, it is determined that, at the first depth of the nerve, the diaphragm is not stimulated and the return signals are not valid, then the method 600 flows “NO” to operation 608. At operation 608, electrodes are placed at a second depth of the nerve. The second depth is deeper than the first depth (e.g., closer to the diaphragm).

Similar to determination 604, at determination 610, it is determined if, at the second depth of the nerve, the diaphragm is stimulated and/or return signals are valid. If it is determined that, at the second depth of the nerve, the diaphragm is stimulated and/or the return signals are valid (e.g., the nerve is intact), then the method 600 flows “YES” to operation 612. At operation 612, it is determined that the nerve is intact at and below the second depth of the nerve and damaged between the first depth and the second depth.

If, alternatively, it is determined that, at the second depth of the nerve, the diaphragm is not stimulated and the return signals are not valid, then the method 600 may either repeat operation 608 to move the electrode even closer to the diaphragm or flow “NO” to operation 614. Operations 608-610 may repeat as required or desired for multiple other depths of the electrodes between the first depth and a maximum stimulation depth (e.g., as described at operation 614). For example, electrodes may be placed at a third depth, a fourth depth, a fifth depth, etc.

At operation 614, electrodes are placed at a maximum stimulation depth of the nerve. For example, for a right phrenic nerve (e.g., phrenic nerve 104A) the maximum stimulation depth may be just above a heart of the patient (e.g., nerve depth B) for a stimulation device placed in a vein or just above the diaphragm (e.g., nerve depth C) for a stimulation device in an esophagus of the patient. For a left phrenic nerve (e.g., phrenic nerve 104B), the maximum stimulation depth may be either just above the heart for an intravenous stimulation device (e.g., nerve depth G) or along a middle portion of the SVC (e.g., nerve depth F) for a stimulation device placed in the esophagus.

Again, like determination 604 and determination 610, at determination 616, it is determined if, at the maximum stimulation depth of the nerve, the diaphragm is stimulated and/or return signals are valid. If it is determined that, at the maximum stimulation depth of the nerve, the diaphragm is stimulated and/or the return signals are valid (e.g., the nerve is intact), then the method 600 flows “YES” to operation 618. At operation 618, it is determined that the nerve is intact at and below the maximum stimulation depth of the nerve and damaged between the second depth and the maximum stimulation depth.

If, alternatively, it is determined that, at the maximum stimulation depth of the nerve, the diaphragm is not stimulated and the return signals are not valid, then the method 600 flows “NO” to operation 620. At operation 620, it is determined that the nerve is damaged at or below the maximum stimulation depth.

At operation 622, information associated with a status of the nerve is provided. The status of the nerve may include which nerve, if damage is detected, and at what depths the nerve damage is detected. For example, a status of the nerve associated with operation 606 may include which nerve and that the nerve is intact at and below the first depth. A status of the nerve associated with operation 612 may include which nerve and that the nerve is intact at and below the second depth and damaged between the first depth and the second depth (or damaged above the second depth). A status of the nerve associated with operation 618 may include which nerve and that the nerve is intact at and below the maximum stimulation depth and damaged between the second depth and the maximum stimulation depth (or damaged above the maximum stimulation depth). A status of the nerve associated with operation 620 may include which nerve and that the nerve is damaged below the maximum stimulation depth.

The information provided with the status of the nerve may also include feasibility of phrenic nerve stimulation therapy and/or a specific stimulation device to administer the therapy. For example, for nerve damage below the maximum stimulation depth, as determined at operation 620, phrenic nerve stimulation therapy may not be available for the patient, or only invasive options with direct access to the diaphragm may be useable. In another example, if the nerve is intact at or below a second depth or maximum stimulation depth that is only achievable with a specific stimulation device (e.g., intravenous stimulation device or esophageal stimulation device), then that device may be specified. For instance, an intravenous stimulation device may be specified for a nerve depth G in FIG. 4. Alternatively, an esophageal stimulation device may be specified for a nerve depth C in FIG. 4.

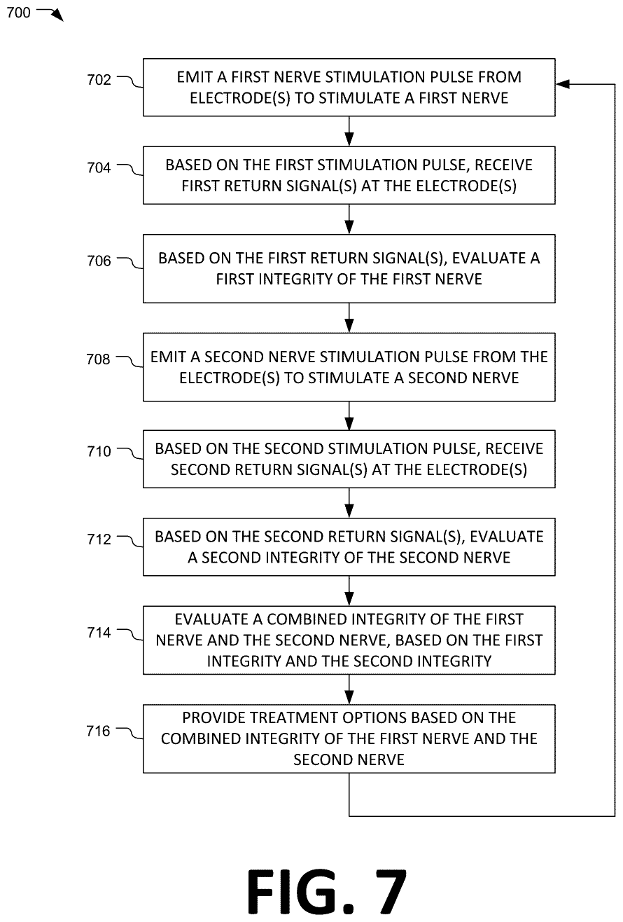

FIG. 7 shows another method 700 for assessing integrity of a nerve. Method 700 determines, in part, if after placing the stimulation device (e.g., according to one or more operations of method 600 in FIG. 6) phrenic nerve stimulation is capable of providing enough therapy to a patient.

At operation 702, a first nerve stimulation pulse is emitted from electrodes to stimulation a first nerve. As described herein, the first nerve stimulation pulse maybe emitted from one or more electrodes of a stimulation device. The first nerve stimulation pulse may have attributes, such as an amplitude, voltage, frequency, duration, etc. The attributes of the first nerve stimulation pulse may be independently controllable. Additionally or alternatively, one or more attributes may vary over time.

At operation 704, a first set of return signals is received at the electrodes, based on the first stimulation pulse. The electrodes receiving the first set of return signals may be the same or different than the electrodes emitting a first nerve stimulation pulse. The first set of return signals includes at least one return signal. A quantity of return signals may indicate if a nerve and/or its branches are intact. In an example, the first set of return signals includes one return signal if the nerve is broken or damaged before any branches. Alternatively, the first set of return signals includes multiple return signals for nerves that are intact or at least one branch is intact.

At operation 706, a first integrity of the first nerve is evaluated, based on the first set of return signals. The first integrity of the first nerve may be evaluated based on attributes of the first set of return signals, such as a time lapse, a return signal band, a conduction, and/or a quantity of return signals. A time lapse may be measured between a time that the first nerve stimulation pulse is emitted and a time that a first return signal of the first set of return signals is received. If a time lapse is too short, then the first nerve may be determined to be damaged or broken. A nerve that is damaged or broken may have no integrity (e.g., an integrity value of zero). A return signal band may be measured between a time that the first return signal and the last return signal of the first set of return signals is received. A band that is too narrow may indicate branch damage of the first nerve. Narrower bands have less integrity than wider bands. Similarly, a populous band (e.g., a greater quantity of return signals, which may also be a wider return signal band) has a greater integrity than a sparse band (e.g., fewer of return signals, which may also be a narrower return signal band). A conduction may be measured for the first set of return signals. Higher conduction values are associated with better integrity and lower conduction values are associated with worse integrity of the first nerve.

In some examples, the nerve integrity may be based on a tidal volume caused by delivering the first nerve stimulation pulse. The integrity may be further evaluated by normalizing the tidal volume by predicted body weight of the patient. By observing and considering effect on tidal volume, respiratory mechanics of the patient are weighed along with nerve damage. For example, a patient with a higher lung compliance may need or desire less stimulation of a first nerve (or may tolerate a first nerve with more damage) to obtain the same normalized tidal volume as a patient with lower lung compliance.

At operation 708, a second nerve stimulation pulse is emitted from the electrodes to stimulate a second nerve. At operation 710, a second set of return signals is received at the electrodes, based on the second stimulation pulse. At operation 712, a second integrity of the second nerve is evaluated, based on the second set of return signals. Operations 708-712 may be similar to operations 702-706, except applied to a second nerve.

At operation 714, a combined integrity of the first nerve and the second nerve is evaluated, based on the first integrity and the second integrity. Full stimulation of both the first nerve and the second nerve may not be necessary for a patient to obtain a target minimum normalized tidal volume. In some situations, stimulation of one intact nerve may provide a patient with at least up to 70% of their desired tidal volume. This is described at least in U.S. patent application Ser. No. 16/888,960, titled “Achieving Smooth Breathing by Modified Bilateral Phrenic Nerve Pacing,” and filed on Jun. 1, 2020, which is mentioned above and incorporated herein by reference in its entirety. Thus, phrenic nerve stimulation therapy may be feasible in situations where one or both nerves are damaged (e.g., which includes situations where one or both nerves is intact). The combined integrity of the first nerve and the second nerve may be evaluated to determine if the patient may be treated solely with phrenic nerve stimulation therapy.

At operation 716, treatment information is provided, based on the combined integrity of the first nerve and the second nerve. For example, treatment information may include the integrity of the first nerve and/or the second nerve and/or the combined integrity of both nerves. A comparison of the integrity of the first nerve and integrity of the second nerve may also be made. For example, if the integrity of each nerve is substantially different (e.g., a difference between the integrity values is at least a threshold value, such as when the nerves have different amounts of damage and return different conduction values), stimulation of both nerves may result in an uncoordinated effort by the patient. Information regarding each nerve's integrity and a difference in the integrities may also be provided. Additionally, an associated amount to desynchronize stimulation of each nerve to coordinate the effort may also be provided.

Additionally or alternatively, the treatment information may include whether or not phrenic nerve stimulation (PNS) therapy is feasible for the patient (e.g., whether or not PNS is a treatment option for the patient) and/or a status of the nerves (e.g., as described at least with respect to FIG. 6). For instance, treatment information may include PNS therapy by stimulating one nerve (e.g., the first nerve or the second nerve may be specified), PNS therapy by stimulating both nerves, PNS therapy by stimulating one nerve (e.g., one nerve is broken or damaged) in combination with positive pressure ventilation from a ventilator, PNS therapy by stimulating both nerves (e.g., at least one nerve is damaged and/or the patient has low lung compliance) in combination with positive pressure ventilation from a ventilator, or no PNS therapy and only positive pressure ventilation from a ventilator. Other treatment information is possible, such as desynchronized PNS therapy between the two nerves, a recommendation to image the diaphragm (e.g., an ultrasound image), etc.

Operations 702-716 may repeat as required or desired. For example, operations may repeat for a different location of the electrodes in the body of the patient. Additionally, the method 700 may be repeated at different times during a patient's therapy. For instance, in some situations a nerve may heal or otherwise repair damage over time, thus resulting in increased integrity. Treatment information may therefore change over time.

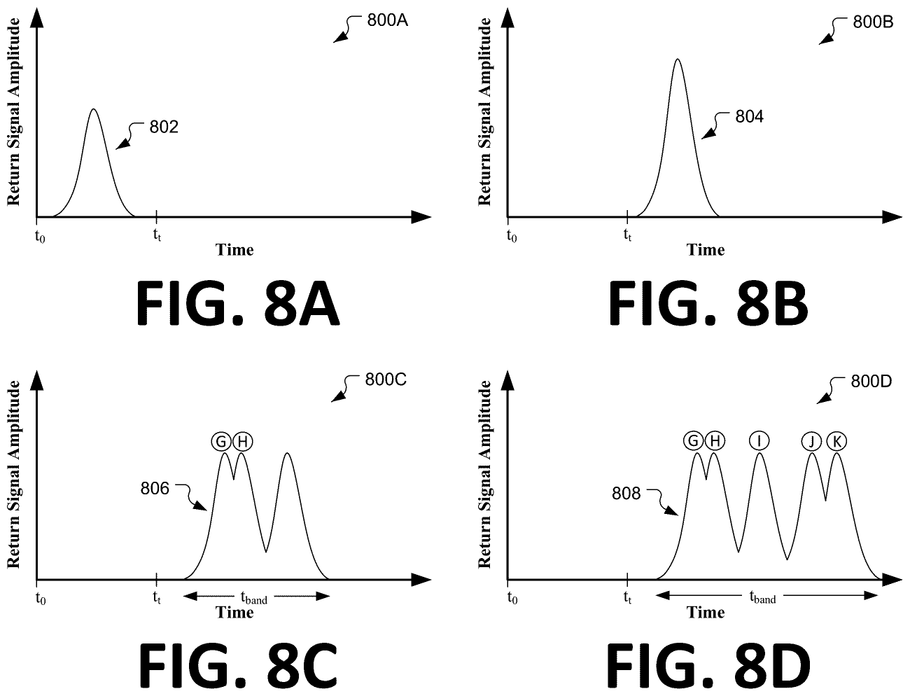

FIGS. 8A-8D show graphical representations 800A-D of return signal(s) of a nerve over time as originating from a stimulation pulse. In particular, FIG. 8A shows a graphical representation 800A of a return signal 802 associated with a nerve that is broken before the diaphragm (e.g., before the branches of the nerve). FIG. 8B shows a graphical representation 800B of a return signal 804 associated with a nerve that is broken or damaged at the diaphragm. FIG. 8C shows a graphical representation 800C of a return signal 806 associated with a nerve that includes some branches that are intact and some that are damaged. FIG. 8D shows a graphical representation 800D of a return signal 808 associated with a nerve that is intact. Each of the graphical representations 800A-D include a stimulation time, to, at which a nerve stimulation pulse is delivered, and a threshold time, tₜ, associated with a nerve depth of the nerve stimulation pulse. As described herein, a return signal measured prior to the threshold time tₜ indicates nerve damage prior to the diaphragm.

As shown in FIGS. 8A-8D, the return signal may have different amplitudes, which may be associated with conduction of the nerve. For example, the amplitude of the return signal 804 in FIG. 8B is greater than the amplitude of the return signal 802 in FIG. 8A. Higher amplitudes (e.g., higher conductivity) is associated with greater integrity of the nerve (e.g., greater diaphragm stimulation achieved via stimulating the nerve).

Each peak in the return signal indicates either a branch end (e.g., branch ends H-Q in FIG. 4) or a break or damage along the nerve. A single peak, such as that shown in FIGS. 8A-8B, may indicate nerve damage prior to an end of any branch of the nerve. Single peaks before the threshold time tₜ are breaks or damage above the diaphragm (e.g., as shown in FIG. 8A). Single peaks after the threshold time tₜ are breaks or damage at or along the diaphragm.

Multiple peaks are shown in the return signals 806, 808 in FIGS. 8C-8D. As described herein, the time between the first peak and the last peak of the return signal is the return signal band t_{band}. Wider bands t_{band} may indicate more branches are intact (e.g., more populous) and narrower bands team indicate less branches are intact (e.g., more sparse). In the example shown in FIG. 8C, two branches are intact (e.g., branch ends G-H in FIG. 4 reflected back the return signal 806) and there is damage along a third branch (e.g., damage prior to branch end I in FIG. 4). In the example shown in FIG. 8D, all branches are intact (e.g., branch ends G-K in FIG. 4 reflected back the return signal 808).

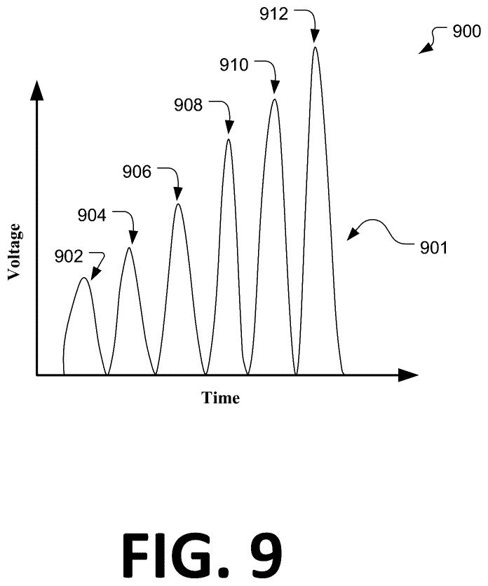

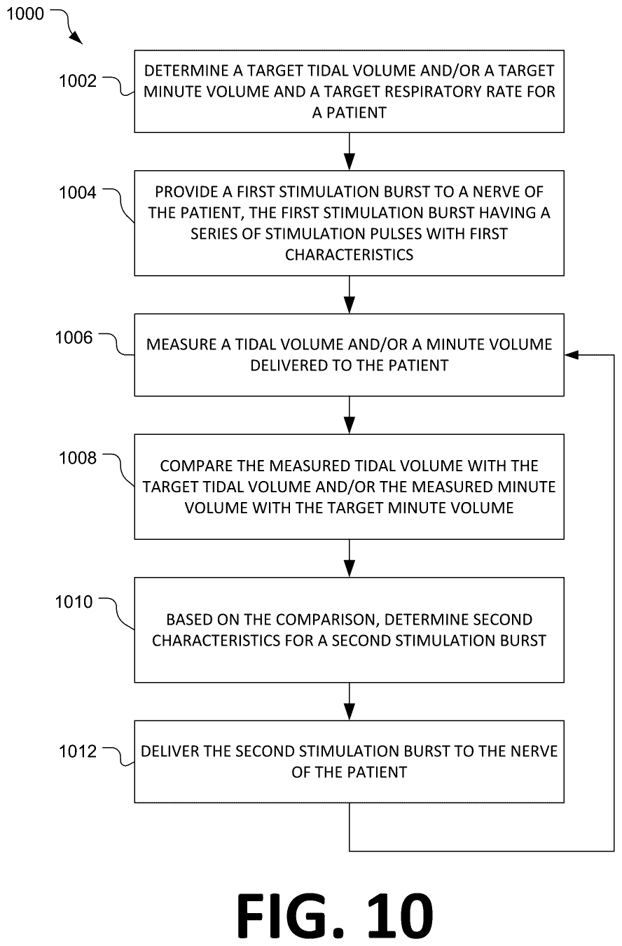

When beginning to administer nerve stimulation therapy, the diaphragm may be unprepared for certain amounts of stimulation, which could sometimes cause hiccups or other discomfort to the patient. In addition to assessing nerve integrity of the patient, a stimulation burst may be delivered as a ramped-up pulse string (e.g., a series of pulses with increasing voltages) to achieve smooth breathing of the patient. FIGS. 9 and 10 describe ramping up stimulation pulses in a stimulation burst.

FIG. 9 shows a graphical representation 900 of a ramped-up stimulation burst 901 voltage over time. As shown in FIG. 9, a stimulation burst 901 includes a pulse string with a series of stimulation pulses 902-912. Although six stimulation pulses 902-912 are shown for the stimulation burst 901 in FIG. 9, any number of stimulation pulses during a stimulation burst is appreciated (e.g., 2, 3, 4, 5, 7 or more). The initial stimulation pulse 902 has a lower voltage than the second stimulation pulse 904, the second stimulation pulse 904 has a lower voltage than the third stimulation pulse 906, which has a lower voltage than the fourth stimulation pulse 908, etc., such that each pulse in the pulse string of the stimulation burst 901 has increased voltage from the prior pulse in the pulse string. Thus, the stimulation burst 901 includes a series of stimulation pulses 902-912 with increasing voltage from an initial stimulation pulse 902 to a final stimulation pulse 912. The stimulation burst 901 (e.g., having the pulse string of stimulation pulses 902-912) is delivered during an inhalation phase of a breath of a patient.

The stimulation burst 901 may have a burst duration appropriate to match an inhalation phase of the patient, such as 1-2 seconds. No stimulation burst may be delivered during an exhalation phase of the patient, which may last approximately 2 seconds. For example, the stimulation burst 901 may be delivered during a first inhalation phase for 1-2 seconds, then paused during exhalation, and then repeated during a subsequent inhalation phase for another 1-2 seconds. The burst duration of the stimulation burst 901 may be selected or varied to achieve a target respiratory rate (e.g., 15-20 breaths/min). The target respiratory rate may include a maximum respiratory rate to prevent or reduce overstimulation of the diaphragm. For example, if stimulation bursts 901 are provided to a patient in fast succession, the diaphragm may become overstimulated and may cause damage to the diaphragm or lungs and/or cause discomfort to the patient.

Characteristics of the stimulation burst 901, including the burst duration, quantity of pulses, initial and final voltages of the pulses, relative voltages of the pulses, and frequency of the pulses, may vary from patient-to-patient to achieve a desired or target tidal volume and/or minute volume. In an example, a target tidal volume for a patient is 6-8 mL/kg. For example, to increase a tidal volume caused by administering the stimulation burst 901, the burst duration, quantity of stimulation pulses 902-912, voltage of the initial stimulation pulse 902, voltage of the final stimulation pulse 912, rate of increase of the voltages between the initial stimulation pulse 902 and the final stimulation pulse 912 of the stimulation burst 901 (e.g., slope of the pulse string), and/or frequency of the stimulation pulses 902-912 may be increased. Alternatively, to decrease a tidal volume caused by administering the stimulation burst 901, the burst duration, quantity of stimulation pulses 902-912, voltage of the initial stimulation pulse 902, voltage of the final stimulation pulse 912, rate of increase of the voltages between the initial stimulation pulse 902 and the final stimulation pulse 912 of the stimulation burst 901 (e.g., slope of the pulse string), and/or frequency of the stimulation pulses 902-912 may be decreased. Changes to the characteristics of the stimulation burst 901 may be based on respiratory rate in addition to tidal volume and/or minute volume for safety of the patient (e.g., reduce or prevent overstimulation of the diaphragm).

Diaphragms of different patients may respond differently to a stimulation burst 901 with matching characteristics. Attributes of a patient that may influence reactivity to a stimulation burst include age, lung compliance, diaphragm atrophy, nerve integrity (e.g., as described herein), etc. For example, patients who are older, have lower lung compliance, and/or have limited nerve integrity may require a greater contractual force of the diaphragm to achieve similar tidal volumes to patients who are younger, have higher lung compliance, and/or have good nerve integrity. Additionally, patients with weaker diaphragms and/or a diaphragm that is experiencing muscle atrophy may desire less stimulation to prevent or reduce injury to the diaphragm.

Further, characteristics of a stimulation burst 901 may be varied depending on how much tidal volume is already being achieved from a patient's effort. A tidal volume portion attributable to a patient's effort may be determined using a ventilator-initiated procedure, such as a negative inspiratory force (NIF) maneuver. Based on the tidal volume portion attributable to a patient's effort, characteristics of the stimulation burst 901 may be varied or modified accordingly to achieve a target total tidal volume. For example, the tidal volume attributable to the stimulation burst 901 may be adjusted lower for greater patient efforts and the tidal volume attributable to the stimulation burst 901 may be adjusted higher for lesser patient efforts. Additionally, characteristics of the stimulation burst 901 may be adjusted based on known information about the patient, such as medical history, prior ventilation data, imaging of the diaphragm of the patient, etc.

A voltage of an initial stimulation pulse 902 and a final stimulation pulse 912 may be set or determined based on a target tidal volume of the patient. For example, the voltage of the final stimulation pulse 912 may be a voltage that results in the patient achieving the target tidal volume (e.g., 100% of a stimulation voltage associated with achieving the target tidal volume for the patient). In another example, the voltage of the initial stimulation pulse 902 may be a voltage that results in the patient achieving less than half of the target tidal volume (e.g., 20%, 25%, 30%, etc. of a stimulation voltage associated with achieving the target tidal volume for the patient). Additionally or alternatively, the initial stimulation pulse 902 may be a percentage of the final stimulation pulse 912 (e.g., 20%, 25%, 30%, etc.). Alternatively, the voltage of the initial stimulation pulse 902 may be a preset value. The preset value may be a low voltage (e.g., a voltage substantially below a voltage that would achieve a target tidal volume for the patient) to avoid a physiological response of the patient, such as a hiccup.

The voltages of the stimulation pulses 904-910 between the initial stimulation pulse 902 and a final stimulation pulse 912 may increase in voltage value according to a pattern (e.g., a rate of voltage increase of the stimulation pulses 902-912 in the pulse string or slope of the pulse string). For example, increase in voltage from pulse-to-pulse in the stimulation burst may be linear, exponential, logarithmic, etc. In another example, the voltage of an initial stimulation pulse 902 may double by the third stimulation pulse 906 or fourth stimulation pulse 908. In a further example, the voltage of a initial stimulation pulse 902 may triple by the fifth stimulation pulse 910 or sixth stimulation pulse 912.

A pulse duration for each stimulation pulse 902-912 in the stimulation burst 901 may be the same. Additionally, the frequency of the stimulation pulses 902-912 in the stimulation burst 901 may be predetermined. The frequency of the stimulation pulses 902-912 may be between seconds and 0.4 seconds. In a specific example, the frequency of the stimulation pulses 902-912 may be between 20-80 Hz (e.g., 40 Hz), with a pulse width between 200-800 μs (e.g., 500 μs). As described above, the stimulation burst 901 may be cycled every 1-2 seconds.

FIG. 10 shows a method 1000 for a ramped-up a stimulation burst (e.g., stimulation burst 901 in FIG. 9) to stimulate a nerve (e.g., a right phrenic nerve and/or a left phrenic nerve). At operation 1002, a target tidal volume and/or a minute volume is determined for the patient. A respiratory rate may also be determined for the patient. The target tidal volume and/or the target minute volume may be based on a patient information, such as age, gender, ethnicity, predicted body weight, medical history, prior ventilation data, imaging of the diaphragm of the patient, etc. In an example, the target tidal volume may be between 6-8 mL/kg of predicted body weight. A target respiratory rate may be determined for the patient to prevent or reduce overstimulation of the diaphragm. In an example, the target respiratory rate is between 15-20 breaths/min.

At operation 1004, a first stimulation burst is provided to a nerve (e.g., a phrenic nerve). The first stimulation burst may be provided during a first inhalation of the patient. As described herein, the first stimulation burst may include a pulse string having a series of stimulation pulses with first characteristics. Characteristics of the stimulation pulses may include an initial stimulation pulse voltage (e.g., a voltage of the initial stimulation pulse in the pulse string), a final stimulation pulse voltage (e.g., a voltage of the final stimulation pulse in the pulse string), a quantity of stimulation pulses, a rate of change of the pulse string, a pulse duration of each stimulation pulse, a frequency of the stimulation pulses, etc. In an example, the first characteristics may be predetermined based on the target tidal volume and/or target minute volume and target respiratory rate. For example, at least the voltage values of the initial and final stimulation pulses may be based on the target tidal volume and at least the pulse duration, frequency, and quantity of stimulation pulses (e.g., which influence a total burst duration of the stimulation pulse) may be based on the target respiratory rate. The first characteristics may be different from patient-to-patient, based on patient information. For example, the initial voltage of the first stimulation burst may be higher for patients with greater predicted body weight or patients that are older. As another example, the initial voltage of the first stimulation burst may be lower for patients with greater predicted body weight, known diaphragm atrophy, or younger.

At operation 1006, a tidal volume delivered to the patient is measured. Additionally or alternatively, a minute volume delivered to the patient is measured. The tidal volume and/or minute volume may be measured by a ventilator.

At operation 1008, the measured tidal volume is compared with the desired tidal volume. Additionally or alternatively, the measured minute volume is compared with the desired minute volume. The comparison may be performed by a ventilator. The comparison may occur prior to the next, consecutive inhalation of the patient, such as in real time and/or during an exhalation phase following the first inhalation.

Based on the comparison, at operation 1010, second characteristics are determined for a second stimulation burst. One or more of the characteristics between the first stimulation burst and the second stimulation burst may be different. For example, a change may be made for one or more of the initial stimulation pulse voltage, the final stimulation pulse voltage, the quantity of stimulation pulses, the rate of change of the pulse string, the pulse duration of each stimulation pulse, the frequency of the stimulation pulses, etc. In some instances, one or more of the characteristics may be the same or remain unchanged. Changes in characteristics may be based on a difference between the measured and target values. For example, an amount to increase the voltage of the final stimulation pulse may be based on a difference in the measured and target tidal volume. Other parameters may also be monitored and compared, such as changes in rise time.

At operation 1012, the second stimulation burst is delivered during a second inhalation of the patient. The second inhalation is subsequent to the first inhalation. In an example, the second inhalation is the next, consecutive inhalation of the patient during the next breath. Delivery of stimulation bursts occur during inhalation of the patient. Stimulation burst may occur during each inhalation phase over a treatment period, or may skip some inhalations during a treatment period. For example, the first stimulation burst may be delivered during a first breath and the second stimulation burst may be delivered during a second breath.

Operations 1006-1012 may repeat as required or desired. For example, the stimulation bursts may be delivered for an inhalation of a third breath, a fourth breath, etc. The characteristics of the stimulation pulses of the stimulation bursts may be updated based on measured tidal volume and/or measured minute volume from breath-to-breath or after a certain number of breaths.

FIG. 11 is a diagram illustrating an example of a ventilator 1100 connected to a patient 1150 and a stimulation device. Ventilator 1100 includes a pneumatic system 1102 (also referred to as a pressure generating system 1102) for circulating breathing gases to and from patient 1150 via the ventilation tubing system 1130, which couples the patient to the pneumatic system via an invasive (e.g., endotracheal tube, as shown) or a non-invasive (e.g., nasal mask) patient interface.

Ventilation tubing system 1130 may be a two-limb (shown) or a one-limb circuit for carrying gases to and from the patient 1150. In a two-limb example, a fitting, typically referred to as a “wye-fitting” 1170, may be provided to couple a patient interface 1180 to an inhalation limb 1134 and an exhalation limb 1132 of the ventilation tubing system 1130.

Pneumatic system 1102 may have a variety of configurations. In the present example, pneumatic system 1102 includes an exhalation module 1108 coupled with the exhalation limb 1132 and an inhalation module 1104 coupled with the inhalation limb 1134. Compressors or other source(s) of pressurized gases (e.g., air, oxygen, and/or helium) are coupled with inhalation module 1104 to provide a gas source for ventilatory support via inhalation limb 1134. Stimulation control module 1118 may provide voltage to a stimulation device (e.g., to independently control electrodes on the stimulation device) as described herein. The pneumatic system 1102 may include a variety of other components, including mixing modules, valves, sensors, tubing, accumulators, filters, etc.

Controller 1110 is operatively coupled with pneumatic system 1102, signal measurement and acquisition systems, and an operator interface 1120 that may enable an operator to interact with the ventilator 1100 (e.g., change ventilator settings, select operational modes, view monitored parameters, etc.). Controller 1110 may include memory 1112, one or more processors 1116, storage 1114, and/or other components of the type found in command and control computing devices. In the depicted example, operator interface 1120 includes a display 1122 that may be touch-sensitive and/or voice-activated, enabling the display 1122 to serve both as an input and output device.

The memory 1112 includes non-transitory, computer-readable storage media that stores software that is executed by the processor 1116 and which controls the operation of the ventilator 1100. In an example, the memory 1112 includes one or more solid-state storage devices such as flash memory chips. The processor 1116 may be configured to control attributes of the electrodes on a stimulation device. In an alternative example, the memory 1112 may be mass storage connected to the processor 1116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 1116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

FIG. 12 is a block-diagram illustrating an example of a ventilatory system 1200. Ventilatory system 1200 includes ventilator 1202 with its various modules and components. That is, ventilator 1202 may further include, among other things, memory 1208, one or more processors 1206, user interface 1210, and ventilation module 1212 (which may further include an inhalation module 1214 and an exhalation module 1216). Memory 1208 is defined as described above for memory 1112. Similarly, the one or more processors 1206 are defined as described above for one or more processors 1106. Processors 1206 may further be configured with a clock whereby elapsed time may be monitored by the ventilatory system 1200.

The ventilatory system 1200 may also include a display module 1204 communicatively coupled to ventilator 1202. Display module 1220 (otherwise referred to as operator interface 1210) provides various input screens, for receiving input, and various display screens, for presenting useful information. Inputs may be received from a clinician. The display module 1220 is configured to communicate with user interface 1210 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, patient information, parameter settings, modes, etc.). The elements may include controls, graphics, charts, tool bars, input fields, icons, etc. Alternatively, other suitable means of communication with the ventilator 1202 may be provided, for instance by a wheel, keyboard, mouse, or other suitable interactive device. Thus, user interface 1210 may accept commands and input through display module 1204. Display module 1204 may also provide useful information in the form of various ventilatory data regarding the physical condition of a patient and/or a prescribed respiratory treatment. The useful information may be derived by the ventilator 1202, based on data collected by a data processing module 1222, and the useful information may be displayed in the form of graphs, wave representations (e.g., a waveform), pie graphs, numbers, or other suitable forms of graphic display. For example, the data processing module 1222 may be operative to determine ventilation settings (otherwise referred to as ventilatory settings, or ventilator settings) associated with a stimulation device for nerve stimulation, etc., as detailed herein.

Ventilation module 1212 may oversee ventilation of a patient according to ventilation settings. Ventilation settings may include any appropriate input for configuring the ventilator to deliver breathable gases to a particular patient, including measurements and settings associated with exhalation flow of the breathing circuit. Ventilation settings may be entered, e.g., by a clinician based on a prescribed treatment protocol for the particular patient, or automatically generated by the ventilator, e.g., based on attributes (i.e., age, diagnosis, ideal body weight, predicted body weight, gender, ethnicity, etc.) of the particular patient according to any appropriate standard protocol or otherwise, such as may be determined in association with stimulating a phrenic nerve with a stimulation device. In some cases, certain ventilation settings may be adjusted based on the exhalation flow, e.g., to optimize the prescribed treatment.

Ventilation module 1212 may further include an inhalation module 1214 configured to deliver gases to the patient and an exhalation module 1216 configured to receive exhalation gases from the patient, according to ventilation settings that may be based on the exhalation flow. As described herein, inhalation module 1214 may correspond to the inhalation module 1104, 1214, or may be otherwise coupled to source(s) of pressurized gases (e.g., air, oxygen, and/or helium), and may deliver gases to the patient. As further described herein, exhalation module 1116 may correspond to the exhalation module 1108 and 1216, or may be otherwise coupled to gases existing the breathing circuit.

FIG. 13 is an example stimulation system 1300 including a stimulation device 1302, an application tool 1304, and a connector 1306. The stimulation device 1302 shares aspects with the stimulation devices described herein (e.g., stimulation device 202). The application tool 1304 is any tool known in the art capable of assisting with inserting, implanting, removing, or otherwise moving the stimulation device within the body of the patient while the application tool 1304 remains outside of the body of the patient. The application tool 1304 enables a stylet to be inserted into, removed from, or otherwise moved inside of the stimulation device 1302 to cause the stimulation device 1302 to change shape and/or be secured inside of the body of the patient.

The connector 1306 may be any connector known in the art capable of including independent leads for one or more of the electrodes on the stimulation device 1302. Each lead of the connector 1306 may independently energize the electrode to which the lead is electrically coupled. For example, if the stimulation device has eight electrodes, the connector 1306 may have eight independent leads electrically coupled to each of the eight electrodes. The connector 1306 may be electrically couplable to a controller to independently energize each electrode on the stimulation device 1302 at each lead. The controller may be a component of a ventilator (e.g., stimulation lead control module 1118 of ventilator 1102). Alternatively, the controller may be a stand-alone nerve stimulation controller or may be incorporated into some other device such as a patient monitor.

FIG. 14 is another example stimulation system 1400 including a stimulation device 1402, an application tool 1406, and a connector 1408. The stimulation device 1402 shares aspects with the stimulation device described herein (e.g., stimulation device 302). The application tool 1406 is any tool known in the art capable of assisting with inserting, implanting, removing, or otherwise moving and/or securing the stimulation device 1402 within the body of the patient while the application tool 1406 remains outside of the body of the patient. The application tool 1406 enables inflation and deflation of an inflatable balloon of the stimulation device (e.g., to secure the stimulation device 1402 inside the body of the patient).

The connector 1408 may be any connector known in the art capable of including independent leads (e.g., wires) for one or more of the electrodes on the stimulation device 1402. The connector 1408 may couple to a power port of a connector 1404 of the stimulation device 1402. Each lead of the connector 1408 may independently energize the electrode to which the lead is electrically coupled. For example, if the stimulation device 1402 has eight electrodes, the connector 1408 may have eight independent leads electrically coupled to each of the eight electrodes. The connector 1408 may be electrically couplable to a controller to independently energize each electrode on the stimulation device 1402 at each lead. The controller may be a component of a ventilator (e.g., stimulation control module 1118 of ventilator 1100). Alternatively, the controller may be a stand-alone nerve stimulation controller or may be incorporated into some other device such as a patient monitor. If an external pad is used, the same or different controller and/or connector 1408 may be used to control electrodes in the external pad.

Although the present disclosure discusses the implementation of these techniques in the context of phrenic nerve stimulation, stimulation of any nerve or nerves in the body of a patient is appreciated. Additionally, although the stimulation devices are described with specific uses and functions for nerve stimulation, any device with one or more leads may be used. Additionally, any lead with a potential to damage a nerve during a medical procedure may be used to test the nerve for damage before, during, and/or after the procedure. For example, a cardiac ablation tool may be used in lieu of the stimulation devices described.

Additionally, although placement of the stimulation devices is described with respect to veins and an esophagus of a patient, placement of the stimulation device in any orifice in the body is appreciated. For example, the stimulation device can be adapted for any vein or tube in the body to excite any nerve nearby.

Further, the techniques introduced above may be implemented for a variety of medical devices or devices utilizing nerve stimulation. A person of skill in the art will understand that the technology described in the context of a medical ventilator for human patients could be adapted for use with other systems such as ventilators for non-human patients or general gas transport systems. Additionally, a person of ordinary skill in the art will understand that the modeled exhalation flow may be implemented in a variety of breathing circuit setups.

Those skilled in the art will recognize that the methods and systems of the present disclosure may be implemented in many manners and as such are not to be limited by the foregoing aspects and examples. In other words, functional elements being performed by a single component or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method for determining phrenic nerve integrity, the method comprising:
positioning a set of electrodes inside a body of a patient at a location near a phrenic nerve;
emitting a nerve stimulation pulse from the set of electrodes to stimulate the phrenic nerve;
receiving, at the set of electrodes, a return signal based on the nerve stimulation pulse, the return signal having a first signal and a last signal;
measuring a return signal band between the first signal and the last signal; and
based on the return signal band, determining an integrity of the phrenic nerve.

2. The method of claim 1, wherein positioning the set of electrodes inside the body includes:
placing the set of electrodes at a first nerve depth of the phrenic nerve;
measuring a first time lapse value between the nerve stimulation pulse and the first signal of the return signal at the first nerve depth, wherein the first time lapse value is less than a threshold value;
placing the set of electrodes at a second nerve depth of the phrenic nerve; and
measuring a second time lapse value between the nerve stimulation pulse and the first signal of the return signal at the second nerve depth, wherein the second time lapse value is at least the threshold value.

3. The method of claim 2, based on the first time lapse and the second time lapse, determining a presence of nerve damage of the phrenic nerve between the first nerve depth and the second nerve depth.

4. The method of claim 2, wherein the set of electrodes is positioned inside of a vein or an esophagus of the patient.

5. The method of claim 1, wherein determining the integrity of the phrenic nerve includes determining, based on the return signal band, that nerve damage is located along at least one branch of the phrenic nerve.

6. A method for determining phrenic nerve integrity, the method comprising:
emitting a nerve stimulation pulse from a first electrode at a nerve depth to stimulate a phrenic nerve of a patient;
based on the nerve stimulation pulse, receiving a return signal at a second electrode at the nerve depth, wherein the return signal includes at least a first signal and a last signal, wherein a time lapse is defined between the nerve stimulation pulse and the first signal of the return signal, and wherein a return signal band is defined between the first signal and the last signal;
based on the time lapse and the return signal band, determining an integrity of the phrenic nerve; and
determining treatment information for the patient, based on the integrity of the phrenic nerve.

7. The method of claim 6, wherein the integrity of the phrenic nerve includes a location of nerve damage along the phrenic nerve.

8. The method of claim 6, wherein the phrenic nerve is a first phrenic nerve and the integrity is a first integrity, and wherein the method further includes:
determining a second integrity of a second phrenic nerve; and
determining a combined integrity of the first phrenic nerve and the second phrenic nerve based on the first integrity and the second integrity, wherein the treatment information is based on the combined integrity.

9. The method of claim 8, wherein the treatment information includes one of:
a feasibility of phrenic nerve stimulation therapy;
the first integrity of the first phrenic nerve;
the second integrity of the second phrenic nerve; or
the combined integrity.

10. The method of claim 6, wherein the method further includes:
displaying the treatment information.

11. A method for ramping up nerve stimulation, the method comprising:
providing, from a stimulation device, a stimulation burst to a patient, the stimulation burst including:
an initial stimulation pulse with an initial voltage;
a final stimulation pulse with a final voltage; and
at least one middle stimulation pulse between the initial stimulation pulse and the final stimulation pulse, the middle stimulation pulse having at least one middle voltage greater than the initial voltage and less than the final voltage.

12. The method of claim 11, wherein the initial stimulation pulse, the at least one middle stimulation pulse, and the final stimulation pulse are all emitted within less than three seconds.

13. The method of claim 11, wherein the stimulation burst is a first stimulation burst, the method further comprising:
measuring a tidal volume delivered to the patient according to the first stimulation burst;
comparing the measured tidal volume with a target tidal volume; and
based on the comparison, delivering, from the stimulation device, a second stimulation burst including a second initial stimulation pulse with a second initial voltage and a second final stimulation pulse with a second final voltage, wherein the second final voltage is higher than the second initial voltage and higher than the final voltage of the first stimulation burst.

14. The method of claim 11, wherein a slope defined by the initial voltage, the at least one middle voltage, and the final voltage is linear.

15. The method of claim 11, wherein the initial voltage is less than 30% of the final voltage.
